(19) United States
(12) Patent Application Publication
(10) Pub. No.: US 2026/0273211 A1
(43) Pub. Date: Sep. 17, 2026

(54) ASSISTED BREATHING APPARATUS AND METHOD

(71) Applicant: ARIA METASYSTEMS S.A.P.I. DE C.V., Ciudad de Mexico (MX)

(72) Inventor: Julio Alberto Rosano Garcia, Puebla (MX)

(73) Assignee: ARIA METASYSTEMS S.A.P.I. DE C.V., Ciudad de Mexico (MX)

(21) Appl. No.: 19/139,416

(22) PCT Filed: Dec. 13, 2023
(86) PCT No.: PCT/MX2023/050072

§ 371 Date: Jun. 16, 2025

(30) Foreign Application Priority Data
Dec. 16, 2022 (MX) MX/A/2022/016601

(51) Int. Cl. A61M 16/00; A61M 16/16; A61M 16/20

(52) U.S. Cl. CPC A61M 16/024; A61M 16/161; A61M 16/202; A61M 2205/3327; A61M 2205/3331; A61M 2230/432

(57) ABSTRACT

A respiratory assistance apparatus that provides respiratory support to patients who are unable to breathe on their own or experience difficulty doing so, having a series of electronic, mechanical, and control arrangements to perform such actions, supplying a constant flow of air/oxygen to the patient.

### TECHNICAL FIELD

The present invention pertains to the technical field of human needs, more specifically to the field of health and medical sciences; and even more particularly to devices for introducing or administering gases into the human body. In an even more specific aspect, it relates to devices for influencing the respiratory system of patients through gas-based treatment by stimulating respiratory movement using mechanical, pneumatic, or electrical means. Still more specifically, the present invention relates to an assisted breathing apparatus and method.

### BACKGROUND

Currently, various devices, tools, and apparatuses are disclosed in the state of the art, all aimed at providing a means of respiratory assistance for patients experiencing difficulty breathing. These devices, commonly referred to as ventilators, generally correspond to mechanical ventilators that incorporate a means for delivering airflow and oxygen, typically with low inertia and variable speeds, specifically adapted to provide the full functionality of a ventilator. The prior art includes a wide range of mechanical ventilators for patients requiring respiratory assistance.

Unfortunately, such mechanical ventilators have traditionally been configured as large devices that occupy a relatively significant portion of the limited space available in operating rooms and patient recovery areas. Moreover, these mechanical ventilators from the prior art are typically inefficient, requiring large amounts of energy to operate. In addition, the prior art mechanical ventilators have not been truly portable devices in the sense that they possess relatively high weight and volume, which limits their portability.

Furthermore, in general, mechanical ventilators have achieved improved functionality in terms of their ability to deliver various breathing modes to the patient by using rotary drag compressors that operate at low pressure to administer breathing. These drag compressors may operate in variable-speed or constant-speed modes.

Mechanical ventilators operating in variable-speed mode provide inspiratory support (i.e., inhalation assistance) to a patient by rapidly accelerating the compressor from a standstill, followed by rapid deceleration during the expiratory phase (i.e., exhalation) of the breathing cycle. Unfortunately, such rapid acceleration and deceleration require complex drive circuitry for the compressor and involve high electrical current consumption. The current draw of such variable-speed drag compressors is relatively high, which increases the overall cost of the mechanical ventilator. Moreover, the high current requirement necessitates the incorporation of bulky and heavy batteries to provide backup battery power as an emergency supply when the ventilator is not connected to a stationary power source or to an arrangement that may include a different drive element, such as a turbine-compressor unit.

Alternatively, rotary drag compressors may operate in constant-speed mode to eliminate the limitations imposed by the high current requirements of variable-speed compressors. Unfortunately, such constant-speed drag compressors have their own inherent shortcomings that reduce the overall utility of the mechanical ventilator. For example, since the compressor runs at a constant speed, power is continuously consumed even during the expiratory phase (i.e., exhalation) when no air or gas is being delivered to the patient. Although energy consumption may be reduced by recirculating the airflow during exhalation back to the compressor intake, a considerable amount of backup battery power is still required to operate the mechanical ventilator when it is not connected to a stationary power source.

As can be observed, there is a need in the art for a mechanical ventilator that is small in size and lightweight to enhance its portability. In addition, there is a need in the art for a portable mechanical ventilator capable of providing respiratory assistance to a patient over extended periods without the limitations of a stationary power source. Furthermore, there is a need in the art for a portable mechanical ventilator that can deliver respiratory assistance in both volume-control and pressure-control modes and that can be operated safely and quietly in noise-sensitive environments such as operating rooms, intensive care units, and patient recovery areas.

Currently, there is no apparatus for this nature capable of recording all the programmed and collected information during its operation.

Such information could be processed and made available online globally to improve medical protocols and procedures.

It is also known that European patent EP 0324275 B1 broadly describes a ventilation synchronizer in which the rising edge of an impedance signal is detected to indicate the onset of spontaneous inspiration, and a gas supply from a respirator is synchronized, through a ventilation synchronizer, with a selected point during the period of spontaneous inspiration. In addition, apnea conditions are detected to activate a backup system that periodically supplies gas to the patient despite the absence of spontaneous breathing. If desired, the triggering and disconnection of the respirator do not need to be synchronized to occur simultaneously with the start of inspiration and exhalation; instead, this can be achieved at selected times after the onset of inspiration or exhalation. The activation and deactivation of the respirator can also be configured in a way that minimizes the effects of a drifting baseline.

Instead of using electrical impedances between two points on a person's chest to indicate spontaneous breathing, other signals may also be used, such as electromyograms, signals indicating contractions of the chest muscles, signals from pneumatic devices indicating abdominal movement, and ultrasound signals indicating movement of the patient's diaphragm.

Another document pertaining to the state of the art of the present invention is the portable mechanical ventilator with drag compressor detailed in U.S. patent application US20030230307A1, which describes a ventilation device and system comprising a rotary compressor, preferably a drag compressor, which, at the beginning of each inspiratory ventilation phase, accelerates to a speed sufficient to deliver the desired inspiratory gas flow, and subsequently stops or decelerates to a baseline flow level to allow the expiratory ventilation phase to occur. The ventilation device is small and lightweight enough to be used in portable applications. The ventilation device is sufficiently efficient to operate for extended periods using internal or external batteries. An oxygen mixing apparatus is also provided, which uses solenoid valves with specific orifice sizes to mix desired amounts of oxygen into the inspiratory gas flow. Additionally, an exhalation valve is provided, featuring an exhalation flow transducer that incorporates a radio frequency database to supply an auxiliary controller with calibration information specific to the exhalation flow transducer.

In the same vein, U.S. Pat. No. 9,925,346B2 discloses ventilation systems and methods involving unknown exhalation flow, wherein systems and methods are provided that allow the patient to trigger or initiate the delivery of a breath. Additionally, systems and methods are disclosed for initiating ventilation when the exhalation flow is unknown or unreliable to the ventilator. Similarly, a series of methods and devices for varying the backup rate of a ventilator are disclosed in U.S. Pat. No. 8,051,852B2, wherein a ventilation device provides ventilatory support to a patient in a timed backup mode when patient breathing is not detected, or in a spontaneous mode when patient breathing is detected. The time threshold governing the backup mode is selected to deviate from the expected normal breathing time of the patient, in order to encourage patient-initiated ventilation in the spontaneous mode while still allowing for backup ventilation in the event of apnea. Automated adjustments to the time threshold during the timed mode are made from a less vigilant time threshold to a more vigilant one at or near the expected normal breathing time of the patient. Such adjustments may be made from a minimum to a maximum vigilance time setting, or incrementally between them, based on the duration of the timed mode, preferably corresponding to the number of machine-delivered breaths.

Finally, the closest document known to the present invention is U.S. patent application US20190143059A1, which discloses patient ventilation systems and methods, wherein a mechanical ventilator is provided that includes a dashboard display identifying the current ventilatory status of a patient within a global or universal ventilatory mechanic's map. This dashboard display is dynamically updated based on the patient's condition and shows trends in the patient's ventilation over time. The map identifies safe and unsafe ventilation regions suggested for the patient, and the ventilator may display informational text, activate audible and/or visual alarms, and transmit alarm communications in response to determining that the patient is approaching or has entered an unsafe region.

The disadvantages found in the prior art arise mainly from the fact that current respirators include an excessive number of components in their assembly, which not only increases their weight but also leads to maintenance procedures that are complex for the personnel responsible for operating and servicing them. Additionally, this complexity increases the risk of component failure, not to mention that both weight and volume are further increased due to the arrangement of the components. Furthermore, there is currently no respirator capable of reliably reading patient parameters under portable operating conditions. These issues result in significant disadvantages for the user, as they prevent the availability of a reliable respirator suitable for use under demanding conditions, with the necessary mechanical and structural robustness to operate without complications. For these reasons, there is a need for a device capable of providing a means of breathing support to patients who require it due to respiratory illness or deficiency, and that can operate without complications for both the patient and the equipment.

Likewise, the greater the number of sensors and solenoid valves in prior art systems, the more time and resources the microprocessor must use to communicate with its peripherals. This is the reason the microprocessor can only process the behavior of each respiratory cycle a limited number of times per second. As a result, necessary corrections to the programmed values occur progressively rather than immediately, which delays such corrections by one or more respiratory cycles before reaching the expected or preset values.

Therefore, the purpose of the present invention is to provide a device and method that addresses the above-described drawbacks and offers significant advantages in its utility. It relates to an assisted breathing apparatus with characteristics analogous to those described in the previously cited documents, but with a structural arrangement of components that allows for simple and easy operation, along with integrated circuits and functions for data processing, without compromising safety in either operability or device performance.

Thanks to the fact that the present assisted breathing apparatus comprises a minimal number of components and sensors, the processing of sensed parameters sent to the microprocessor enables an immediate response. With an average evaluation rate of once per millisecond, the response sent to a solenoid valve to correct the parameter takes only the mechanical reaction time of said component to carry out the correction, which occurs within a fraction of a second.

### BRIEF DESCRIPTION OF THE INVENTION

It is therefore a main object of protection, an assisted breathing apparatus that includes a gas inlet section where there is a normal air inlet, which has a configuration that can be coupled to a normal air promotion means, and an oxygen inlet where both include tightening and sealing means with a wall of the assisted breathing apparatus, and where it also includes an air/oxygen outlet channel that has a termination that can be coupled to an extension to be inserted into a patient through a respiratory assistance accessory during the inspiration process, as well as a control center arranged on an electronic board that contains a circuit capable of executing actions commanded by a microcontroller using commands for passing, blocking, opening, closing, direction of rotation, and interruption or advancement of an electric current for interruption means for switching on or off, where, in addition, certain embodiments can be controlled by position and/or speed commands, and also certain embodiments receive information on position, speed, pressure, amount of oxygen, carbon dioxide, or electric current intensity from a sensor configured for such purposes characterized in that:

The normal air inlet is connected to a normal air solenoid valve that includes one inlet and two outlets, wherein the inlet is connected to the normal air pressure regulator and wherein one of the outlets is coupled to a safety valve and wherein the second outlet of the normal air solenoid valve is connected to a flow control valve that includes a lateral coupling with a stepper motor whose shaft manipulates the opening and closing of said flow control valve and wherein subsequently a regulated normal air conduit is arranged that, through a conduit, is connected to a dual-inlet and single-outlet connector and for oxygen intake it includes an oxygen solenoid valve, which includes one inlet and two outlets, wherein the inlet is connected to the initial oxygen flow conduit and wherein one of the outlets is coupled to a safety valve that allows the discharge or release of the fluid in case a predetermined value is exceeded and wherein the second outlet of the solenoid valve is connected to a flow control valve that includes a lateral coupling with a stepper motor whose shaft manipulates the opening and closing of said flow control valve and wherein subsequently a regulated oxygen conduit is arranged that is coupled to an oxygen sensor connection which receives, at its upper portion, an oxygen sensor, while in continuity with the regulated oxygen conduit a conduit is arranged that is connected to a non-return valve connected to an oxygen conduit that is coupled to a dual-inlet and single-outlet connector which also receives the regulated normal air conduit and whose outlet from said dual-inlet and single-outlet connector is connected to a humidification means that contains water inside and wherein said humidification means conducts outside of its structure the air-oxygen mixture by means of a container outlet conduit that delivers the air-oxygen and humidity mixture into an intermediate conduit, which includes a connection means at one of its ends which, through a coupling means, directs the mixture contained in the container outlet conduit toward a mixing conduit in a perpendicular manner until it reaches a conduit parallel to the intermediate conduit and which is connected to a flow sensor that is connected to a secondary conduction means for the air/oxygen mixture that is directed toward a mixing flow guide that includes an outlet connection for the air/oxygen mixture, wherein said outlet internally includes a Venturi-type flowmeter to measure the flow rate of the air-oxygen mixture, and subsequently the outlet arrangement of the mixture is oriented toward the outside of the assisted breathing apparatus and is coupled to an external flow insertion means.

And finally, in the exhalation process the CO₂-containing fluid is channeled through the air/oxygen mixture outlet in the direction opposite to the inspiration process, wherein the CO₂-containing mixture is channeled toward a CO₂ outlet located in continuous alignment with the linear portion of the mixing flow guide and wherein said CO₂ outlet is interconnected with a CO₂ outlet solenoid valve that finally conducts this gas, through a gas outlet conduit, to a PEEP valve, wherein the latter maintains, by releasing CO₂ into the atmosphere, a positive pressure within the ventilator arrangement and, therefore, a residual volume at the end of exhalation, and wherein the PEEP valve is controlled in its opening and closing level by a PEEP valve servomotor, which in turn is controlled by the microcontroller.

### BRIEF DESCRIPTION OF THE FIGURES

FIG. 1 shows an isometric view of the assisted breathing apparatus (1).

FIG. 2 shows an isometric view of the assisted breathing apparatus (1) connected to a normal air promotion means (6) and an oxygen inlet (7), as well as an outlet with a mask (9a).

FIG. 3 shows an isometric view of the assisted breathing apparatus (1) connected to an air/oxygen supply arrangement and an endotracheal tube (9b).

FIG. 4 shows an isometric view of the assisted breathing apparatus (1).

FIG. 5 shows an isometric view of the assisted breathing apparatus (1).

FIG. 6 shows an isometric view of the assisted breathing apparatus (1).

FIG. 7 shows an isometric view of the assisted breathing apparatus (1).

FIG. 8 shows an isometric view of the assisted breathing apparatus (1).

FIG. 9 shows an isometric view of the normal air intake arrangement of the assisted breathing apparatus (1).

FIG. 10 shows an exploded isometric view of the normal air intake arrangement of the assisted breathing apparatus (1).

FIG. 11 shows an isometric view of the oxygen intake arrangement of the assisted breathing apparatus (1).

FIG. 12 shows an exploded isometric view of the oxygen intake arrangement of the assisted breathing apparatus (1).

FIG. 13 shows an isometric view of the assisted breathing apparatus (1) without the outer casing.

FIG. 14 shows an isometric view of the oxygen and normal air intake arrangement of the assisted breathing apparatus (1).

FIG. 15 shows an isometric view of the assisted breathing apparatus (1) without the outer casing.

FIG. 16 shows an isometric view of the assisted breathing apparatus (1) without the outer casing.

FIG. 17 shows an isometric view of the assisted breathing apparatus (1) without the outer casing.

FIG. 18 shows an isometric view of the assisted breathing apparatus (1) without the outer casing.

FIG. 19 shows a block diagram related to the respiratory assistance method of the invention.

FIG. 20 shows an example of the screen of the assisted breathing apparatus in start menu mode.

FIG. 21 shows an example of the screen of the assisted breathing apparatus during the programming of Tidal Volume.

FIG. 22 shows an example of the screen of the assisted breathing apparatus during the programming of positive end-expiratory pressure.

FIG. 23 shows an example of the screen of the assisted breathing apparatus during the programming of the pressure trigger.

FIG. 24 shows an example of the screen of the assisted breathing apparatus during the programming of the inspired oxygen fraction.

FIG. 25 shows an example of the screen of the assisted breathing apparatus during the programming of the respiratory rate.

FIG. 26 shows an example of the screen of the assisted breathing apparatus during the programming of the inspiration/expiration ratio.

FIG. 27 shows an example of the screen of the assisted breathing apparatus.

FIG. 28 shows an example of the screen of the assisted breathing apparatus for displaying the programmed values.

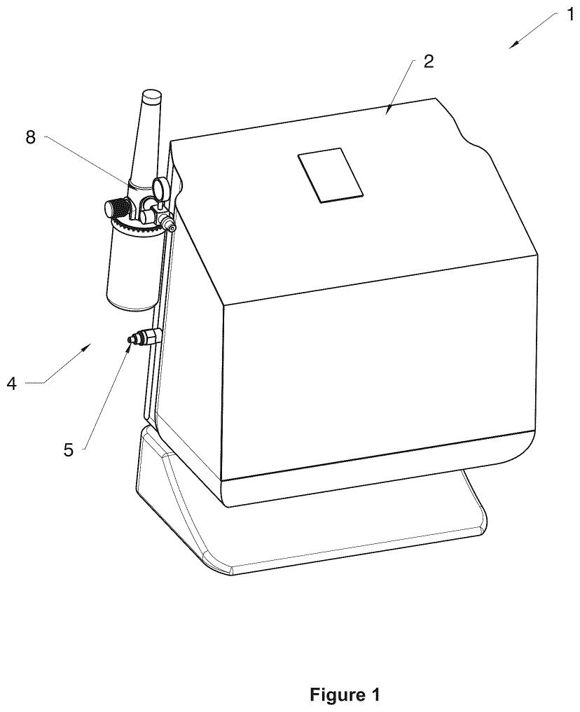

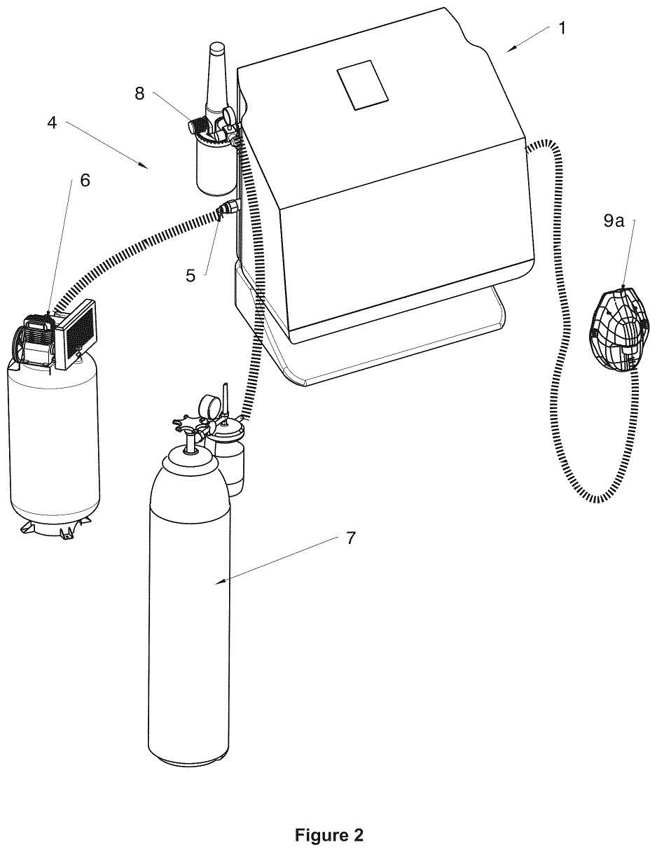

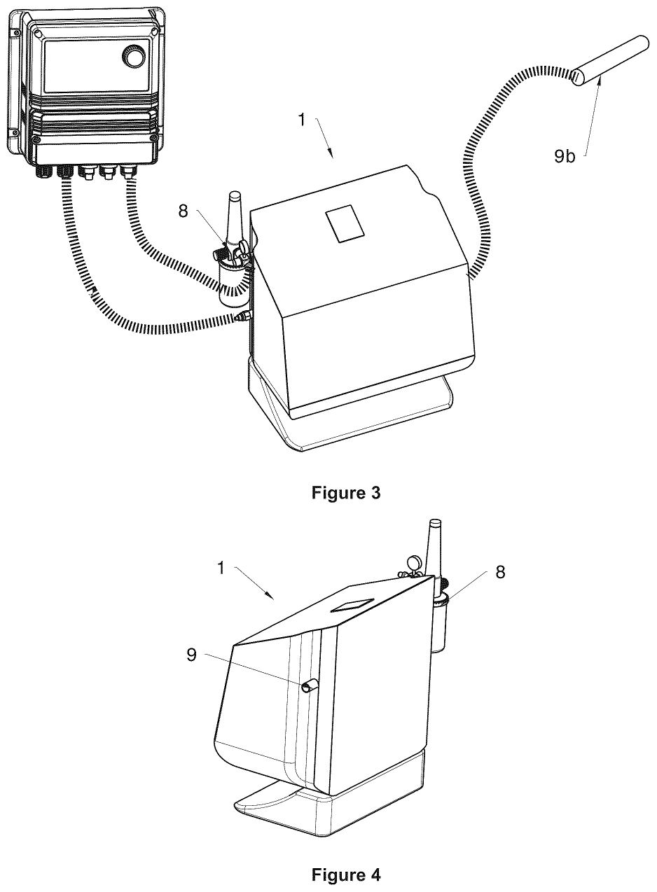

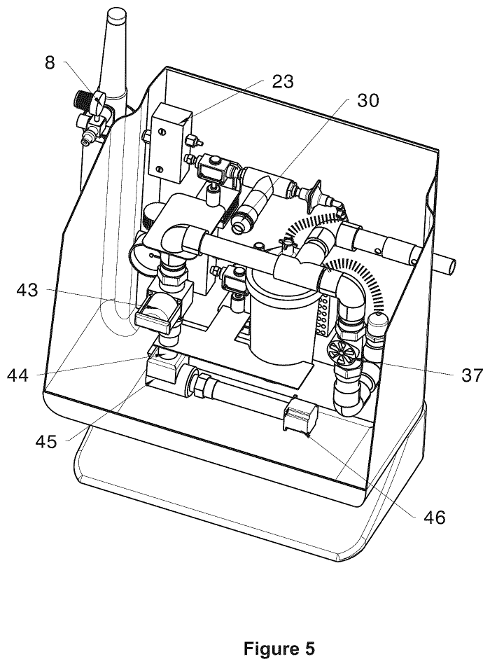

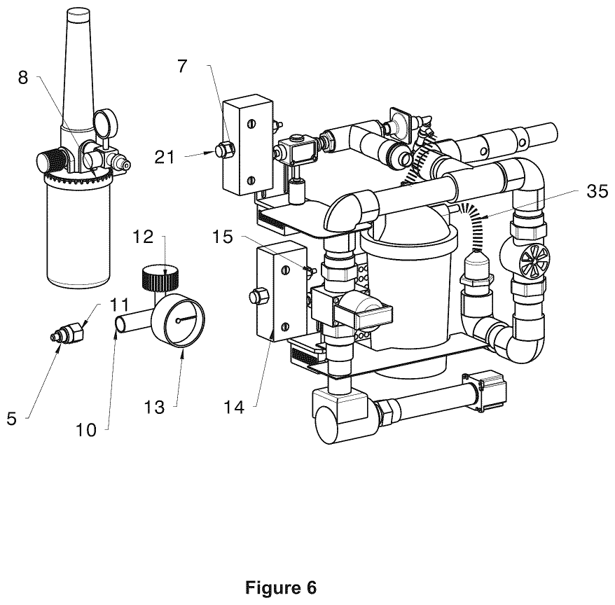

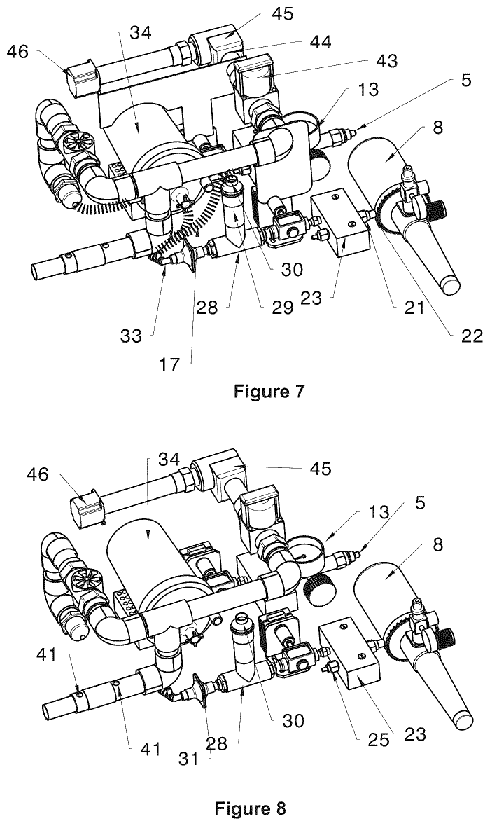

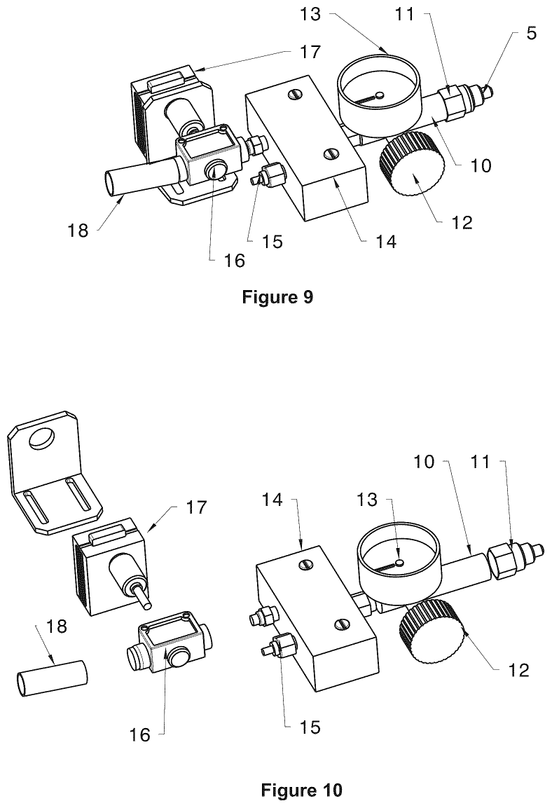

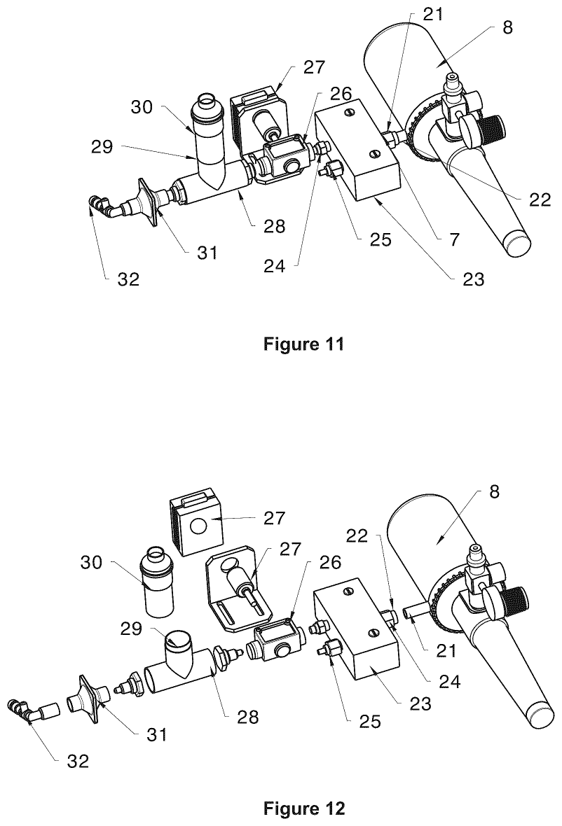

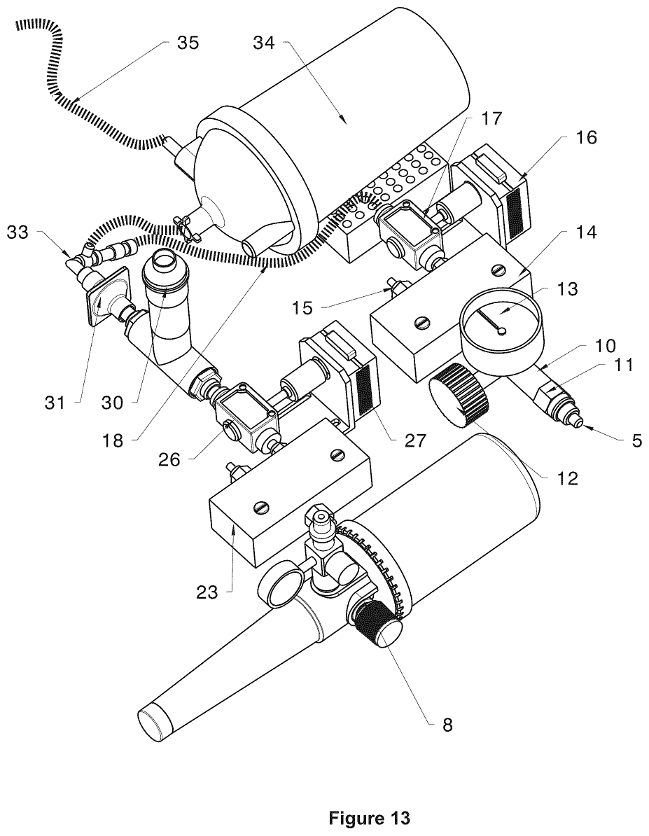

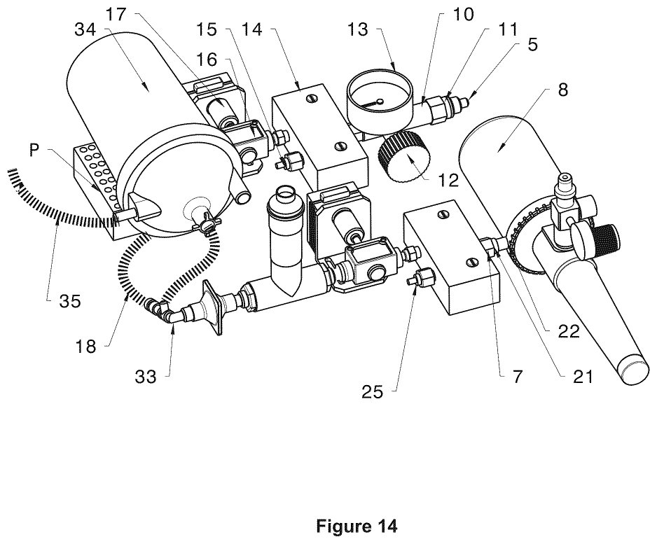

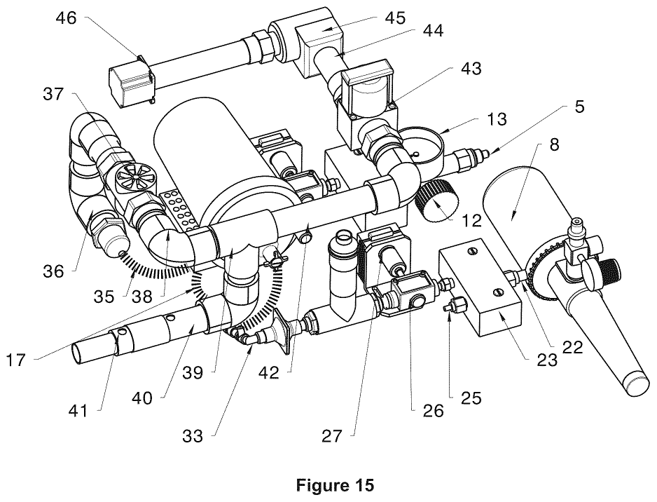

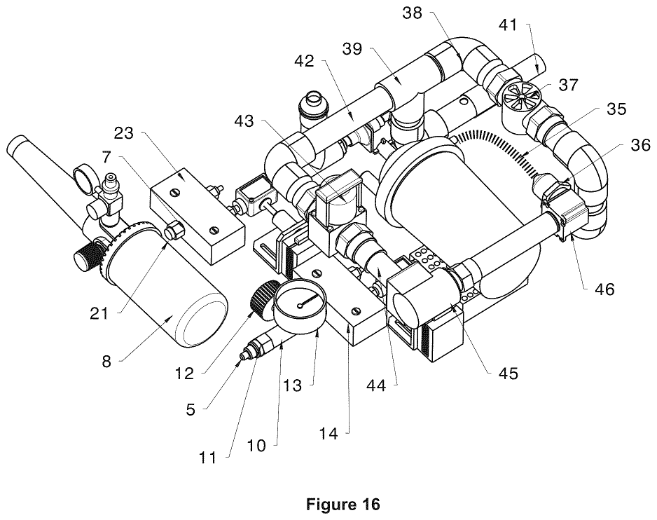

### DESCRIPTION OF THE INVENTION

Unless otherwise defined, all technical and scientific terms used herein have the same meaning as commonly understood by a person skilled in the art to which this invention pertains. The materials, methods, and examples provided in this document are for illustrative purposes only and are not intended to be limiting. Except to the extent necessary or inherent in the processes themselves, no particular order of the steps or stages of the methods and processes described in this disclosure, including the figures, is intended or implied. In many cases, the order of process steps may vary without changing the purpose or effect of the methods described.

Unless specifically stated otherwise, when reference is made to the assisted breathing apparatus (1) or the assistance apparatus, it shall be understood that the intention is to refer to the assisted breathing apparatus that is the subject of the present invention, and as will be apparent from the following discussions, it will be appreciated that throughout the specification discussions, terms such as “conducting means,” “solenoid valve,” “generation,” “flow guide,” “receiving means,” “definition,” or similar are used, wherein these include actions and/or processes controlled by a computer that manipulates and/or transforms data into other data and wherein said data are represented as physical quantities, such as electrical quantities, temperature parameters, pressure, and volumes and/or said data represent physical objects. The terms “electronic board,” “computer,” “processor,” and “controller” must be interpreted broadly to cover any type of electronic device, component, or unit with data processing capabilities, including, by way of non-limiting example, a personal computer, a server, a computer system, a communication device, a processor (e.g., a digital signal processor (DSP) possibly with integrated memory), a microcontroller, a field-programmable gate array (FPGA), an application-specific integrated circuit (ASIC), etc., any other electronic computing device and/or any combination thereof.

The operations in accordance with the teachings of the present document may be performed by a computer or electronic board specially constructed for the desired purposes or by a general-purpose computer specially configured for the desired purpose by a computer-controllable or computer-configurable electronic board and other means or medium stored on a computer-readable storage medium.

As used herein, the phrase “for example,” “such as,” “for instance,” and their variants describe non-limiting embodiments of the subject matter of the present invention. Reference in the specification to “one case,” “some cases,” “other cases,” or variants thereof means that a particular feature, structure, or characteristic described in connection with the embodiment(s) is included in at least one embodiment of the disclosed subject matter. Therefore, the appearance of the phrase “one case,” “some cases,” “other cases,” or variants thereof does not necessarily refer to the same embodiment(s).

It will be appreciated that certain features of the subject matter of the present invention, which are described, for the sake of clarity, in the context of separate embodiments, may also be provided in combination with a single embodiment. Conversely, various features of the subject matter of the present invention, which are described, for reasons of brevity, in the context of a single embodiment, may also be provided separately or in any subcombination.

In order to promote an understanding of the principles of the invention, reference will now be made to a preferred embodiment illustrated in the drawings, and specific language will be used to describe the same. However, it will be understood that no limitation of the scope of the invention is intended, and such further alterations and modifications in the illustrated apparatus, and such further applications of the principles of the invention as illustrated therein, are contemplated as would normally occur now or in the future to one skilled in the art to which the invention pertains.

The present invention refers to an assisted breathing apparatus (1) that provides respiratory support to patients when they are unable to breathe on their own or experience difficulty doing so, in general, such that for its configuration and programming of the respiratory gas flow to the patient, only the following input data are required: the patient's weight expressed in kilograms, the patient's height expressed in centimeters, and the patient's gender (male or female). The assisted breathing apparatus (1) provides a mixture of air/oxygen to be introduced into the patient's lungs, and it is generally composed of a gas inlet section, a gas conduction section inside the apparatus, a gas delivery portion to the patient through a gas expulsion arrangement, and a fluid control and management arrangement contained within the apparatus, wherein it includes a control board comprising a microcontroller that allows the selection of the operating modes of the apparatus, controlled from a user interface that displays a plurality of graphical selection interfaces where the personnel responsible for operating the equipment enter the corresponding values for the required data so that the air/oxygen flow from the apparatus to the patient is optimal, and wherein the operating modes of the apparatus also include a plurality of buttons, knobs, levers, and analogous controls that may be connected to the user interface for modifying the apparatus parameters.

First, a gas inlet section (4) is provided in the assisted breathing apparatus (1), wherein a normal air inlet (5) is arranged, which has a configuration that can be coupled to a normal air promotion means (6), preferably an air compressor, while on the same side of the assisted breathing apparatus (1) there is an oxygen inlet (7), which optionally includes an oxygen regulator (8), and wherein both the normal air inlet (5) and the oxygen inlet (7) include tightening and sealing means with the lateral wall of the assisted breathing apparatus (1) where they are placed, and wherein, in turn, both the normal air inlet (5) and the oxygen inlet (7) are coupled on one of the faces of the assisted breathing apparatus (1) in a parallel manner and preferably in a horizontal arrangement.

At the opposite end of the assisted breathing apparatus (1), where the gas inlet section (4) is located, an air/oxygen outlet channel (9) is also provided, which includes a termination that can be coupled to an extension, wherein once the air/oxygen mixture has completed its path through the interior of the assisted breathing apparatus (1), it is expelled to be delivered to a patient by means of a mask (9a) or an endotracheal tube (9b) during the inspiration process, and wherein likewise, the return of the mixture during the exhalation process is collected through this conduit for a treatment that will be described below.

In a second preferred embodiment of the invention, the assisted breathing apparatus includes connections to air/oxygen supplies that are already installed in medical units, whereby only the normal air inlet (5) channel and the oxygen inlet (7) channel are required to be coupled to said medical unit.

The assisted breathing apparatus (1) also incorporates a control center arranged on an electronic board that contains a programmable integrated circuit, capable of executing the instructions stored in its memory, namely a microcontroller that provides precise control of a series of actuators using commands for stepping, blocking, opening, closing, direction of rotation, and interruption or advancement of an electric current for interruption means for switching on or off, wherein, in addition, certain embodiments may be controlled by position and/or speed commands, and certain embodiments also receive information on position, speed, pressure, amount of oxygen, carbon dioxide, or electric current intensity from a sensor configured for these purposes.

In the same manner, the incorporation of a pair of power supplies is provided. First, the insertion of a 5-volt power supply that powers the logic portion of the circuit, consisting of microcontrollers, interface screens, and elements that require relatively low voltage levels. On the other hand, a 12-volt power supply (P) is provided, which powers the power section of the system, namely solenoid valves, sensors, relays, and switching elements such as the incorporated stepper motors.

For the intake of normal air inlet (5), a primary channel (10) is provided, defined by a preferably cylindrical conduit, hollowed on its internal portion and comprising a connector (11) with an adjustment or tightening arrangement, wherein said connector is continuously connected at one of its ends to the normal air inlet (5) and at the opposite end of said connection to a normal air pressure regulator (12), which is calibrated during the manufacturing or assembly of the apparatus and which in turn includes a pressure indicator (13) to display the readings of the adjusted pressure, wherein said normal air pressure regulator (12) is connected to a normal air solenoid valve (14), which includes one inlet and two outlets, wherein the inlet is connected to the normal air pressure regulator (12) and wherein one of the outlets is coupled to a safety valve (15) that allows the discharge or release of the normal air fluid coming from the primary channel and from the normal air promotion means (6) in the event a predetermined value is exceeded, and wherein the second outlet of the normal air solenoid valve (14) is connected to a flow control valve (16) that includes a lateral coupling with a stepper motor (17) whose shaft manipulates the opening and closing of said flow control valve (16), subsequently a regulated normal air conduit (18) is provided, wherein the fluid containing the necessary pressure parameters is conveyed so that it can be inserted into a dual-inlet and single-outlet connector (32) and subsequently deposited into a humidification means (34).

Whereas for the intake of oxygen, a continuous arrangement is provided from the oxygen inlet (7), comprising an oxygen inlet nozzle (21) that is connected to an initial oxygen flow conduit (22), which at each of its ends includes a threaded rib that is eventually connected at one end to the oxygen inlet nozzle (21) and at the opposite end of said connection to an oxygen solenoid valve (23), which includes one inlet and two outlets, wherein the inlet is connected to the initial oxygen flow conduit (22), and wherein one of the outlets is coupled to a safety valve (25) that allows the discharge or release of the fluid in the event that a predetermined value is exceeded, and wherein the second outlet (24) of the oxygen solenoid valve (23) is connected to a flow control valve (26) that includes a lateral coupling with a stepper motor (27) whose shaft manipulates the opening and closing of said flow control valve (26), subsequently a regulated oxygen conduit (28) is provided, wherein the fluid containing the pressure parameters from the outlet of the oxygen solenoid valve (23) is conveyed and is perpendicularly coupled to an oxygen sensor connection (29), which receives on its upper part an oxygen sensor (30) that detects the oxygen concentration in this portion of the regulated oxygen conduit (28) and sends the reading to the microcontroller on the electronic board. Subsequently, a continuation is provided with a conduit that is connected to a non-return valve (31), which prevents the previously regulated oxygen from returning to the oxygen solenoid valve (23), and in the event that the normal air intake were to reach a pressure parameter with a magnitude greater than that of the oxygen pressure, said non-return valve (31) likewise prevents the return of the mixture, so that once past this non-return valve (31), the oxygen flow can only proceed in one direction toward the outlet of the assisted breathing apparatus (1), and subsequently, at the outlet of the non-return valve (31), a dual-inlet and single-outlet connector (32) is provided, which receives an oxygen conduit (33) connected to the outlet of the non-return valve (31) and a regulated normal air conduit (18) coming from the flow control valve, and the outlet of said dual-inlet and single-outlet connector (32) is connected to a humidification means (34).

In an alternative embodiment of the present invention, an additional air-oxygen mixer or blender (47) is provided, which includes a pair of flow connectors, comprising an air inlet and an oxygen inlet, wherein for the oxygen inlet there is an oxygen conduit (48) coming from the outlet of the non-return valve (31), while the air comes from the regulated normal air conduit (18), and wherein the air-oxygen mixer or blender (47) includes a flow regulator (49) through which the FiO₂ parameter can be further calibrated, and wherein said regulation means may comprise a coupling with a stepper motor or an actuator means that is connected to the electronic board for the opening or closing of the additional air-oxygen mixer or blender (47), which finally integrates an outlet conduit (50) that delivers the air-oxygen mixture into the humidification means (34).

The humidification means (34) refers to a container that contains a portion of water heated to a temperature between 36 and 40° C., said temperature being achieved through thermal transfer from a series of resistive elements housed in a power supply (P) arranged on one side of the humidification means (34), or with said temperature being controlled by a resistive element that generates the required heat to raise the temperature of the water, so that inside the humidification means (34) a mixture of air/oxygen and humidity is developed, which allows for a more complete homogenization, and wherein said mixture is conducted out of the humidification means by means of a container outlet conduit (35) connected at the upper part of the humidification means (34), and which delivers the air-oxygen-humidity mixture into an intermediate conduit (36), which includes a connection means at one of its ends, which, through a coupling means, directs the mixture contained in the container outlet conduit (35) toward a mixing conduit in a perpendicular direction until reaching a conduit parallel to the intermediate conduit (36), which is connected to a flow sensor (37) responsible for detecting the specific amount of liters of air per minute of the mixture based on the patient's condition requirements and which also communicates a signal to the microcontroller to modify the gas flow through the normal air solenoid valve (14) and the oxygen solenoid valve (23). Consequently, the flow sensor (37) is connected to a secondary conduit for air/oxygen mixture (38), which is directed toward a mixture flow guide (39) defined by a T-shaped body comprising a linear conduit and a perpendicular conduit, wherein said perpendicular conduit includes an air/oxygen mixture outlet connection (40), which internally includes a Venturi-type flowmeter (not shown) to measure the flow rate of the air-oxygen mixture, and wherein said Venturi flowmeter includes a pair of conduit receiving means (41) in the form of through holes into which two conduits are respectively inserted and connected to a differential pressure sensor (not shown) located on the electronic board, and wherein this sensor controls the behavior of the normal air solenoid valve (14) and the oxygen solenoid valve (23) in order to allow or restrict the passage of normal air or oxygen as required by the patient, such that this sensor performs the function of monitoring the lung pressures of the patient and the inlet pressures of the air/oxygen mixture; subsequently, the outlet arrangement of the mixture is oriented toward the exterior of the assisted breathing apparatus (1) and is coupled to an external flow insertion means such as a mask (9a) or an endotracheal tube (9b).

When the air/oxygen mixture completes its residence within the body of a patient and gives way to the exit of the fluid from the body, with the presence of CO₂ manifesting in the fluid coming from the lungs during the exhalation process, this fluid is channeled through the air/oxygen mixture outlet connection (40) in the direction opposite to the inspiration process, wherein the CO₂-containing mixture is directed toward a CO₂ outlet (42) located in continuous alignment with the linear portion of the mixture flow guide (39), and wherein said CO₂ outlet is interconnected with a CO₂ outlet solenoid valve (43), which ultimately conducts this gas, through a gas outlet conduit (44), to a PEEP valve (45), wherein the latter maintains, by releasing CO₂ into the atmosphere, a positive pressure within the ventilator arrangement and therefore a residual volume at the end of exhalation, with the purpose of maintaining the alveoli open in order to reduce the risk of lung damage due to alveolar collapse. This PEEP valve (45) is controlled in its opening and closing level by a PEEP valve servomotor (46), which in turn is controlled by the microcontroller. In one embodiment of the present invention, the servomotor (45-46) may be replaced by a stepper motor (not shown).

In a preferred embodiment, the air/oxygen mixture outlet connection (40) internally includes an air quality sensor to monitor the CO₂ during exhalation.

The gas mixture handling process and its delivery to a user is carried out through the microcontroller on the electronic board, which controls the opening and closing of the normal air solenoid valve (14) and the oxygen solenoid valve (23), by means of a double-action relay (not shown), and also controls the CO₂ outlet solenoid valve (43), which regulates the exhalation outlet and is also controlled by the same relay, as follows:

When the microcontroller determines, during the inspiration process, the closing of the CO₂ outlet solenoid valve (43), it opens the normal air solenoid valve (14) and the oxygen solenoid valve (23), allowing a previously programmed amount of air and oxygen to pass toward the patient. In this way, by closing the CO₂ outlet solenoid valve (43), pressure loss through the outlet is prevented, thereby ensuring that the air and oxygen mixture directed to the patient can only exit through the air/oxygen mixture outlet connection (40).

At the point of exhalation, the microcontroller closes the arrangement of the normal air solenoid valve (14) and the oxygen solenoid valve (23), and opens the CO₂ outlet solenoid valve (43), such that the exhaled air exits through the CO₂ outlet (42) and cannot be diverted or returned through the secondary conduit for air/oxygen mixture (38).

In the control center (not shown), an electronic board is provided that enables precise control of a series of actuators using step/direction commands in the case of the stepper motors (17), (27), and interruption or advancement of electric current for the general powering of the assisted breathing apparatus, and optionally for the switching on or off of the normal air promotion means (6). Moreover, certain embodiments may be controlled through position and/or speed commands, and certain embodiments also receive information on position, speed, pressure, or electric current intensity from a sensor configured for such purposes, such as the oxygen sensor (30) and the differential pressure sensor (not shown).

Thus, for example, certain embodiments allow for the use of a stepper motor control system to operate the stepper motors (17), (27) with high precision, optionally using a relatively large number of steps with relatively small step increments.

An example embodiment provides a control system for the stepper motors (17), (27), comprising:
a first input configured to receive a digital step/direction command signal;
a second input configured to receive a position signal from a position sensor of the continuously rotating stepper motors (17), (27); and
a processing system configured to control the position of the stepper motors (17), (27) based at least in part on the step/direction command signal and the position signal.

An example embodiment provides a control system for the stepper motors (17), (27), comprising: a first input configured to receive:
a digital step/direction command signal;
a second input configured to receive a position signal from a position sensor; and
a processing system configured to control a servo valve based at least in part on the step/direction command signal and the position signal.

An example embodiment provides a method for controlling the stepper motors (17), (27), comprising:
receiving step command pulses;
maintaining a count of the step command pulses;
receiving position information from a position sensor coupled to the stepper motors; and
using the step pulse count and the position information to control a servo valve coupled to the motor, thereby at least partially controlling the stepper motors.

An example embodiment provides computer-executable code stored on a computer-readable memory, which, when executed, is configured to: receive a count of step command pulses; receive position information from an incremental continuous-rotation position sensor coupled to the stepper motors (17), (27); and use the step pulse count and the position information to control the flow control valve (16) and the flow control valve (26) coupled to the stepper motors, thereby at least partially controlling the normal air or oxygen flow in the assisted breathing apparatus.

Certain example embodiments allow for the precise control of an actuator for the stepper motors (17), (27) using step/direction commands, and certain embodiments allow for the use of a stepper motor control system to operate the stepper motors with high precision. In particular, certain optional embodiments allow for controlling/rotating the stepper motors (17), (27) through a defined number of steps.

Certain embodiments of the controller on the electronic board include a controller for the stepper motors (17), (27) and for the solenoid valves, which accept digital control inputs or analog control inputs through a command means, such as a pushbutton, or from a digital-to-analog converter output from a control computer.

Certain embodiments include some or all of the following safety circuits and mechanisms:
valve enables driven by isolated relay and port lockout outputs;
a primary valve enable safety relays are provided for safe shutdown in case of emergency failure; to reduce electrical noise, optionally isolated DC power (e.g., 12 V DC or another voltage level) is provided for the logic and the power supplies of solenoid valves, motors, and sensors during operation.

An example embodiment provides a control system for the normal air promotion means (6), comprising: a first input configured to receive:
a digital on/off command signal;
a second input configured to receive a pressure signal from a pressure sensor; and
a processing system configured to control a servo valve based at least in part on the on/off command signal and the pressure signal.

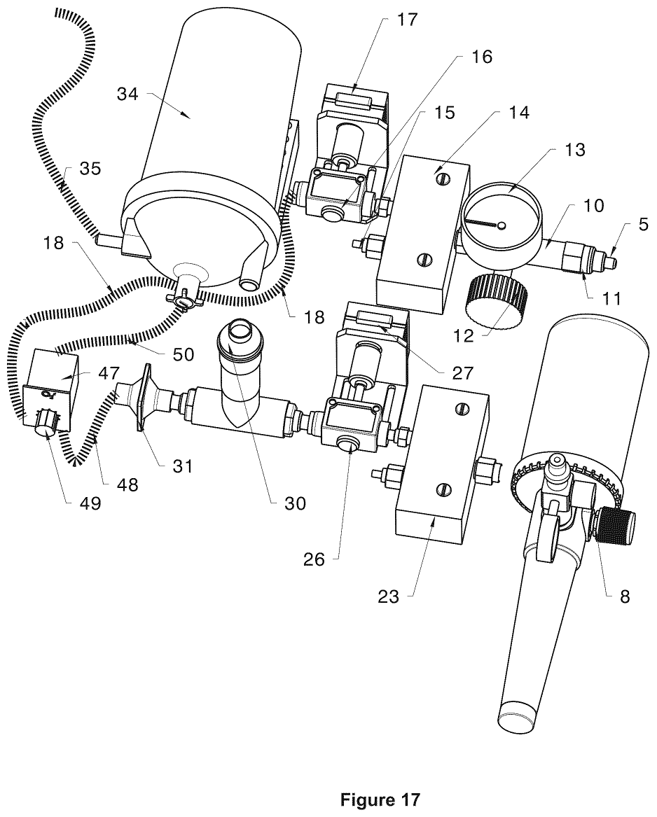

According to FIG. 17, the respiratory assistance method (300) comprises the steps of: receiving an oxygen flow (301a) through or from an oxygen source via an oxygen inlet nozzle; receiving a normal air flow (301b) from a normal air promotion means as a compressed air source through a normal air inlet; regulating the oxygen flow (302a) by means of an oxygen pressure regulator; regulating the air flow (302b) by means of an air pressure regulator; controlling the passage of the regulated oxygen flow (303a) through an oxygen solenoid valve; controlling the passage of the regulated air flow (303b) through an air solenoid valve; presetting (304), through a plurality of graphical user interfaces displayed on a user interface or manipulated via buttons or analogous knobs, the values related to tidal volume, positive end-expiratory pressure, pressure trigger, fraction of inspired oxygen, respiratory rate, and inspiration/expiration ratio according to the patient's needs; mixing (305) the oxygen and air provided by the On/Off oxygen and air solenoid valves; humidifying and increasing the temperature (306) of the air/oxygen/humidity mixture by means of a humidifier; sensing (307) the value related to the number of liters per minute (volume) of the humidified mixture by means of a flow sensor; sensing (308) the value related to the pressure of the inspiratory and expiratory gases through the differential input/output pressure sensor; conducting (309) the humidified mixture at a predetermined pressure and volume toward an air/oxygen outlet channel that delivers it to a patient during the inspiration process; monitoring (310) the CO₂ content in the exhaled gases by means of an exhalation air quality sensor; regulating (311), automatically and in real time, the oxygen pressure supplied to the patient as well as the number of liters per minute (volume) of the humidified mixture.

It should be noted that, under normal conditions, the programmed values should be absolute and constant in order to maintain the patient's respiration in optimal conditions. However, in practice, this is not the case, since the patient's conditions are by no means stable, and due to such variations, the programmed values also become relative and variable.

For this reason, the microprocessor is programmed with control algorithms which a person skilled in the art will understand are not typical of software executed by any conventional computer. These algorithms were specifically conceived for the operation of the apparatus of the present invention, in order to modify said values in real time, without relying on complex equations, simply deciding to increase or decrease pressures, flow, and timing based on the differences between the programmed values and the values sensed in each respiratory cycle, and in this manner, the microprocessor will send corrections to the solenoid valves in order to compensate for any differences that may arise between the programmed values and the actual values required.

Thus, the microprocessor, based on the parameters sensed (approximately 1000 times per second) by the differential pressure and flow sensors during each respiratory cycle, executes the steps of: controlling, by means of the air and oxygen solenoid valves, the flow and pressure of the air and oxygen supplied to the patient in each respiratory cycle based on the preset values related to inspiration, plateau, and expiration, as well as the positive end-expiratory pressure; processing the sensed values related to the pressure of inspiratory and expiratory gases to determine in real time, based on said result, the actual pressure value of the humidified mixture delivered to the patient; processing the sensed values related to the volume of the humidified mixture to determine in real time the volume of the humidified mixture delivered to the patient; processing the sensed values related to the pressure of inspiratory and expiratory gases and determining in real time, based on said result, the actual pressure value of the humidified mixture; processing and determining the values related to the CO₂ content in the exhaled gases in order to define the opening and closing of a CO₂ outlet solenoid valve that expels the gases resulting from the patient's exhalation; determining in real time whether the preset values correspond to the sensed values, which must be absolute and constant in order to maintain the patient's respiration in optimal conditions; sending in real time at least one control signal to the oxygen and/or air solenoid valves in order to compensate for at least one pressure difference between the programmed and the sensed values in each respiratory cycle; sending in real time at least one control signal to the pressure control solenoid valve in order to compensate for at least one difference between the programmed number of liters of air per minute according to the patient's condition and the value sensed in each respiratory cycle; sending in real time at least one control signal to the CO₂ outlet solenoid valve in case the sensed residual volume at the end of expiration is lower than the preset value.

In this manner, when the patient reaches the point of inspiration, the microprocessor closes the CO₂ outlet solenoid valve, opens the air solenoid valve and the oxygen solenoid valve, allowing a previously programmed amount of air/oxygen to pass to the patient, preventing pressure loss through the outlet and thus ensuring that the air/oxygen mixture directed to the patient can only exit through the air/oxygen outlet toward the lungs.

Likewise, when the patient reaches the point of exhalation, the microprocessor closes the arrangement of the air solenoid valve and the oxygen solenoid valve and opens the CO₂ outlet solenoid valve, such that the exhaled air exits through the CO₂ outlet and cannot be diverted or returned through the air/oxygen mixture conduction means.

Additionally, and in a second embodiment of the invention, an oximeter is provided that measures oxygen saturation and heart rate, so the monitored vital signs are:
Respiratory Rate
Heart Rate
Oxygen Saturation in the blood.

Where these are also preferably displayed on the graphical user interface of the screen shown in FIG. 26.

The sequence in the microcontroller is arranged to provide a constant flow of air/oxygen mixture and to control the solenoid valves of the arrangement, for which only the patient's gender and weight expressed in kilograms are required. The steps executed by the microcontroller are as follows:

Measure patient
Calculate predicted weight = (cm − 152.4) × 0.9 + G
G = 50 for MALE
G = 45.5 for FEMALE
PROGRAM VENTILATOR
TIDAL VOL ----------- 6 ml × (predicted weight)
PEEP SaO₂/FiO₂
If PEEP is GREATER than 190 → PEEP = 5 to 8 mmH₂O
If PEEP is LESS than 190 → PEEP = 10 mmH₂O
FiO₂ ----- SaO₂ from 88% to 94%
Trigger 2 cmH₂O · L/min
RR 16 to 20 per minute ------ PaCO₂ normal (35-45) (<60 mmHg)
I:E adjust Flow: Ti ----- 1:2

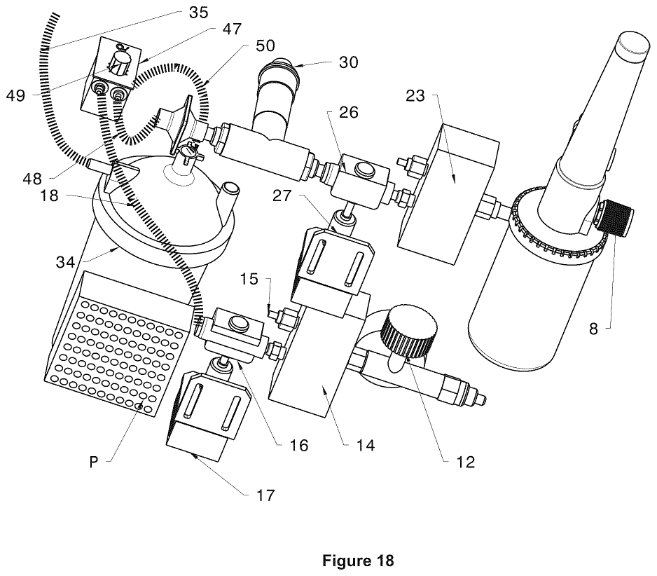

As shown in FIG. 18, a user interface (200) is provided, which, according to the preferred embodiment of the invention, consists of a touchscreen (not limited thereto) that displays a first graphical interface showing the parameters to be monitored within the apparatus: Tidal Volume, Positive End-Expiratory Pressure (PEEP), Pressure Trigger (disp), Fraction of Inspired Oxygen (FiO₂), Respiratory Rate (f), and the Inspiration/Expiration Ratio (I:E), as well as a start button.

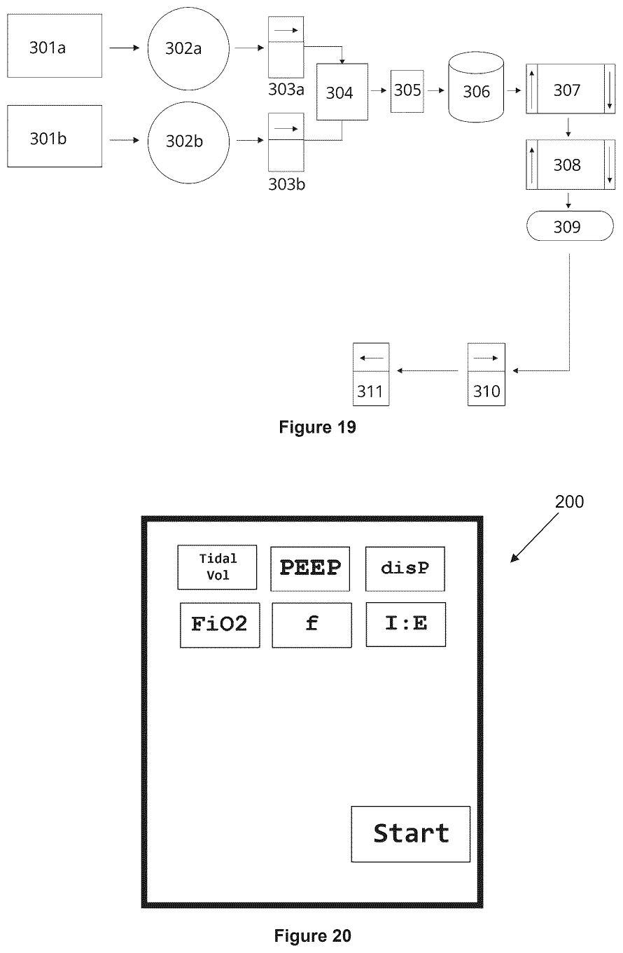

Subsequently, according to FIG. 19, a second graphical interface is provided that displays the user interface (200), in which the following data are entered: gender (201) (MALE or FEMALE), height (202) expressed in centimeters, and weight (203) expressed in kilograms, wherein the input of information may be adjusted using two buttons (204) to increase or decrease the flow generation parameters; once these parameters have been entered, the enter button (205) is pressed.

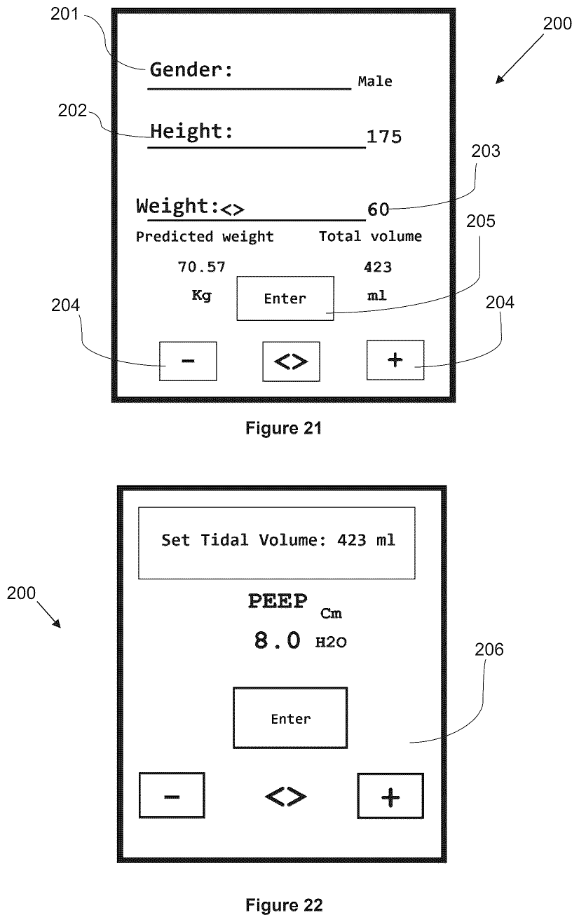

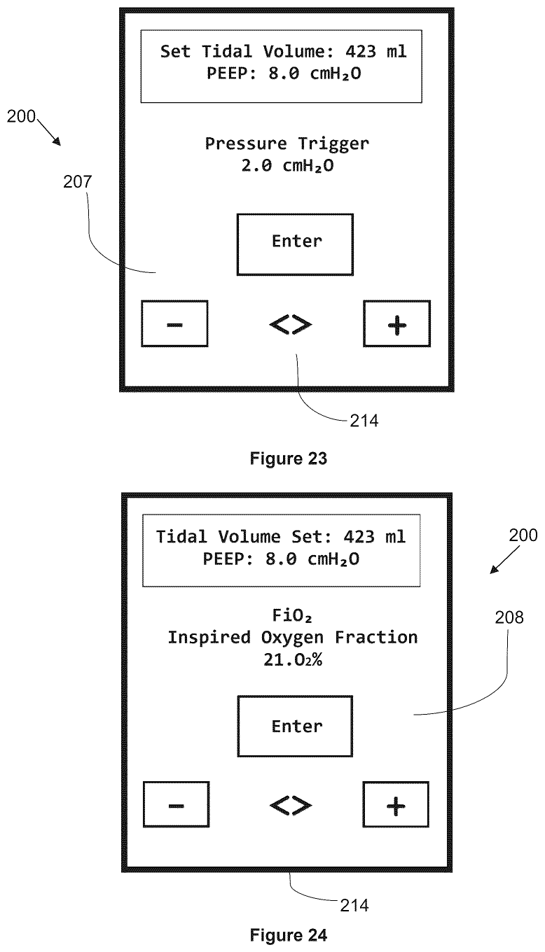

Likewise, FIGS. 20 to 24 show other graphical interfaces displayed by the user interface (200), which eventually display the data for Tidal Volume, Positive End-Expiratory Pressure (PEEP) (206), Pressure Trigger (disp) (207), Fraction of Inspired Oxygen (FiO₂) (208), Respiratory Rate (f) (209), and Inspiration/Expiration Ratio (I:E) (210).

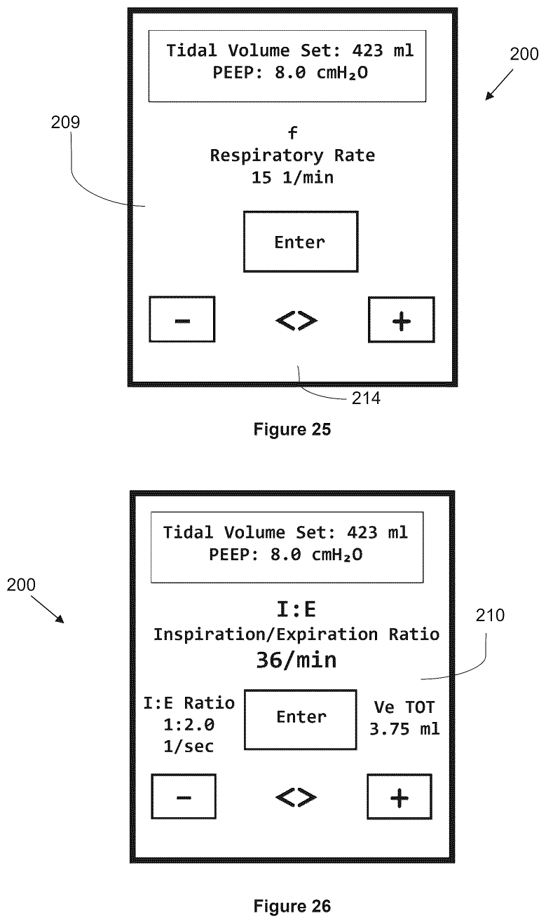

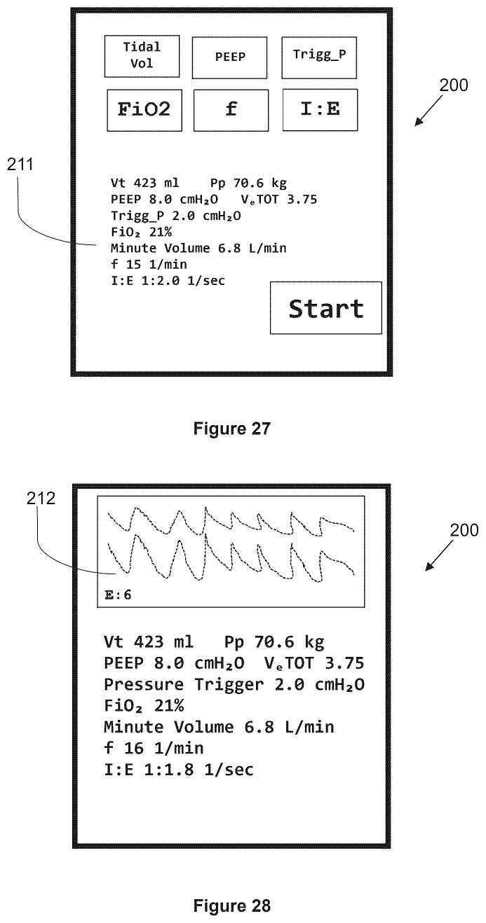

Finally, as shown in FIG. 25, the data are compiled on a screen for final review (211) and the generation of a graph (212), which is displayed to show the patient's respiratory rate, as shown in FIG. 17.

For the selection of parameters in each of the interfaces, selection buttons (214) are provided at the bottom of each interface, allowing variation in the selection of the chosen parameter; these selection buttons are deactivated when their use is not required.

In a third additional embodiment of the invention, an additional circuit is included to provide storage, which is connected either by wired or wireless means to a network through which it provides information to be stored in the cloud, wherein it is integrated by a data storage and distribution unit for transferring such data in order to deposit them into integrated storage, including both local storage and cloud storage; it also includes a storage management unit that connects to the integrated storage in order to organize the data and provide information on the parameters monitored by the assisted breathing apparatus, wherein the storage levels are related to the amount of data stored; it further includes a data handling unit to provide the integrated storage as virtual data storage, regardless of the physical location where the data are stored, and a storage connection unit to provide a user with an interface for virtual data storage as a single virtual storage unit; in addition, this data handling unit provides a write buffer function or a read cache function for the data and reduces the response time of a write operation or a read operation for the data by using high-speed storage.

Wherein the above-described scheme is developed through a method for providing storage to deliver a cloud service, carried out by an apparatus for providing storage to deliver a cloud service, which is integrated by a data distributor to store the data in integrated storage, including local storage and cloud storage; and to manage the data by providing the integrated storage as virtual data storage, regardless of the cloud location of such data.

In a fourth embodiment of the invention, a detection device with an alarm is included, configured to compare physiological data received through the patient monitoring unit, wherein the alarm detection device responds to a predefined measurement of the physiological data such as hypertension or hypotension, abnormal heart rate, hypoxia, or hyperventilation. In turn, the alarm detection device is configured to initiate an alarm comprising at least one speaker with a first frequency, waveform, or duty cycle configured to emit a first alarm signal, and a second alarm signal having a second frequency, waveform, or duty cycle different from the first. Furthermore, an alarm device is provided that emits the second signal if the first signal is emitted and the alarm acknowledgment mechanism is not activated before the specified event, wherein the alarm device is configured to gradually increase the second frequency of the second alarm signal until the second frequency reaches a peak frequency or a notification acknowledgment mechanism is activated.

The system also includes a notification apparatus in the event of a power failure, wherein in addition to activating a backup battery, it comprises an acoustic sensor configured to generate a sensor signal in response to an acoustic, visual, or combined alarm that indicates a system fault condition such as connection failure, electrical failure, among others, and a signal processing unit in communication with the acoustic sensor and configured to generate an identification signal when the acoustic sensor signal satisfies an identification criterion.

Wherein the alarm system is connected to an interface unit in communication with the signal processing unit and configured to generate and send a message signal to a nurse call system of the medical center in response to the identification signal from the signal processing unit.

Although a preferred exemplary embodiment is shown and specified in detail in the foregoing drawings and specification, these should be regarded as purely exemplary and not as limiting of the invention. In this regard, it is noted that only the preferred exemplary embodiment is shown and specified, and all present or future variations and modifications shall be protected within the scope of protection of the invention as defined in the claims.

### LIST OF REFERENCES

assisted breathing apparatus (1)
gas inlet section (4)
normal air inlet (5)
normal air promotion means (6)
oxygen inlet (7)
oxygen regulator (8)
air/oxygen outlet channel (9)
mask (9a)
endotracheal tube (9b)
primary channel (10)
connector (11)
normal air pressure regulator (12)
pressure indicator (13)
normal air solenoid valve (14)
safety valve (15)
flow control valve (16)
stepper motor (17)
regulated normal air conduit (18)
oxygen inlet nozzle (21)
initial oxygen flow conduit (22)
oxygen solenoid valve (23)
second outlet (24)
safety valve (25)
flow control valve (26)
stepper motor (27)
regulated oxygen conduit (28)
oxygen sensor connection (29)
oxygen sensor (30)
non-return valve (31)
dual-inlet single-outlet connector (32)
oxygen conduit (33)
humidification means (34)
container outlet conduit (35)
intermediate conduit (36)
flow sensor (37)
secondary conduit for air/oxygen mixture (38)
mixture flow guide (39)
air/oxygen mixture outlet connection (40)
conduit receiving means (41)
CO₂ outlet (42)
CO₂ outlet solenoid valve (43)
gas outlet conduit (44)
PEEP valve (45)
PEEP valve servomotor (46)
additional air-oxygen blender (47)
oxygen conduit (48)
flow regulator (49)
outlet conduit (50)
power supply (P)

1. An assisted breathing apparatus (1) comprising:
a gas inlet section (4) having an air inlet (5), which is configured to be coupled to an air promotion device (6),
an oxygen inlet (7), both having clamping and sealing devices with a wall of the assisted breathing apparatus (1),
an air/oxygen outlet channel (9) including a termination adapted to be connected to an extension for insertion into a patient through a respiratory assistance accessory during the inspiration process,
a control center arranged on an electronic board containing a circuit capable of executing actions ordered by a microcontroller using step, block, open, close, rotation direction, and interruption or advance commands of an electric current for power-on or power-off interruption device, wherein may be controlled using position and/or speed commands or receive position, speed, pressure, oxygen level, carbon dioxide level, or electric current intensity information from a sensor configured for these purposes,
wherein the air inlet (5) is connected to an air solenoid valve (14) having an inlet and a first outlet and a second outlets, wherein the inlet is connected to the air pressure regulator (12), and the first outlet is coupled to a safety valve (15), and the second outlet of the air solenoid valve (14) is connected to a flow control valve (16), which includes a lateral coupling with a stepper motor (17), whose shaft controls the opening and closing of said flow control valve (16), and wherein a regulated normal air conduit (18) is subsequently provided, which is connected by device a conduit to a dual-inlet single-outlet connector (32),
and for oxygen intake, it comprises an oxygen solenoid valve (23), which has an inlet and two outlets, wherein the inlet is connected to the initial oxygen flow conduit (22), and the first outlet is coupled to a safety valve (25) that allows the discharge or release of fluid in case a predetermined value is exceeded, and wherein the second outlet (24) of the oxygen solenoid valve (23) is connected to a flow control valve (26), which includes a lateral coupling with a stepper motor (27), whose shaft controls the opening and closing of said flow control valve (26), and wherein a regulated oxygen conduit (28) is subsequently provided, which is coupled to an oxygen sensor connection (29) receiving on an upper part an oxygen sensor (30), while in continuation with the regulated oxygen conduit (28), a conduit is arranged that connects to a non-return valve (31), connected to an oxygen conduit (33) that is coupled to a dual-inlet single-outlet connector (32), which also receives the regulated normal air conduit (18), and whose outlet is connected to a humidification device (34) containing water, and wherein said humidification device (34) conducts the air/oxygen mixture outside its structure by device of a container outlet conduit (35), which deposits the air/oxygen and humidity mixture into an intermediate conduit (36), which includes a connection device at one of its ends that, through a coupling device, directs the mixture contained in the container outlet conduit (35) towards a mixing conduit perpendicularly, until reaching a conduit parallel to the intermediate conduit (36), which is connected to a flow sensor (37), which is connected to a secondary conduit for air/oxygen mixture (38), directed toward a mixture flow guide (39), which comprises an air/oxygen mixture outlet connection (40) internally provided with a Venturi-type flowmeter to measure the flow of the air/oxygen mixture, and wherein the mixture outlet arrangement is subsequently directed outside the assisted breathing apparatus (1) and is coupled to an external flow insertion device; and
wherein during the exhalation process, the fluid with CO₂ is channeled through the air/oxygen mixture outlet connection (40) in the opposite direction to the inspiration process, wherein the mixture with CO₂ is directed toward a CO₂ outlet (42), which is continuously arranged with the linear portion of the mixture flow guide (39), and wherein said CO₂ outlet is interconnected with a CO₂ outlet solenoid valve (43), which finally conducts this gas, through a gas outlet conduit (44), to a PEEP valve (45), which maintains, through the release of CO₂ into the atmosphere, a positive pressure in the respirator arrangement and, therefore, a residual volume at the end of exhalation, and wherein the PEEP valve (45) is controlled in its opening and closing level by a PEEP valve servomotor (46), which is in turn controlled by the microcontroller.

2. The assisted breathing apparatus (1) according to claim 1, wherein that the normal air inlet (5) comprises a primary channel (10) defined by a hollow conduit and including a connector (11) with a tightening or clamping arrangement, wherein said connector is continuously connected at one of its ends to the normal air inlet (5) and at the opposite end to a normal air pressure regulator (12), which in turn comprises a pressure indicator (13), and wherein the normal air pressure regulator (12) is connected to the normal air solenoid valve (14).

3. The assisted breathing apparatus (1) according to claim 1, wherein the oxygen inlet comprises an oxygen inlet nozzle (21) connected to an initial oxygen flow conduit (22), and at the opposite end of said connection to the oxygen solenoid valve (23).

4. The assisted breathing apparatus (1) according to claim 1, wherein the oxygen sensor (30) detects the oxygen concentration in the regulated oxygen conduit (28) and sends the reading to the microcontroller of the electronic board.

5. The assisted breathing apparatus (1) according to claim 1, wherein the flow sensor (37) is configured to detect the number of liters per minute of the air-oxygen mixture and transmits a signal to the microcontroller to adjust the gas flow through the normal air solenoid valve (14) and the oxygen solenoid valve (23).

6. The assisted breathing apparatus (1) according to claim 1, wherein further comprising an air-oxygen blender (47) that comprises a pair of flow connectors, including an air inlet and an oxygen inlet, wherein the oxygen inlet is connected to an oxygen conduit (48) that originates from the outlet of the non-return valve (31), while the air inlet is connected to the regulated normal air conduit (18), and wherein the air-oxygen blender (47) includes a flow regulator (49) that allows additional calibration of the FiO₂ parameter, and wherein said flow regulator may comprise a coupling with a stepper motor or an actuator connected to the electronic board for opening or closing the air-oxygen blender (47), which ultimately includes an outlet conduit (50) that delivers the air-oxygen mixture into the humidification device (34).

7. The assisted breathing apparatus (1) according to claim 1, wherein the mixture flow guide (39) is defined by a T-shaped body comprising a linear conduit and a perpendicular conduit.

8. The assisted breathing apparatus (1) according to claim 1, wherein the Venturi flowmeter comprises a pair of conduit receiving device (41) in the form of through bores into which two conduits are respectively inserted, and is connected to a differential pressure sensor located on the electronic board, wherein said sensor controls the operation of the normal air solenoid valve (14) and the oxygen solenoid valve (23) in order to allow or restrict the passage of normal air or oxygen.

9. The assisted breathing apparatus (1) according to claim 1, wherein the PEEP valve servomotor (45) can be replaced by a stepper motor (not shown).

10. The assisted breathing apparatus (1) according to claim 1, wherein further comprising a pair of power supplies, comprising first a 5-volt power supply that powers the logic portion of the circuit including microcontrollers, interface screens, and elements that require relatively low voltage levels, while a 12-volt power supply (P) powers the power portion of the system, namely, solenoid valves, sensors, relays, and opening and closing elements such as the integrated stepper motors.

11. The assisted breathing apparatus (1) according to claim 1, wherein the normal air promotion device (6) is an air compressor, and the oxygen inlet (7) comprises an oxygen regulator.

12. The assisted breathing apparatus (1) according to claim 1, wherein that the respiratory assistance accessory is a mask (9a) or an endotracheal tube (9b).

13. The assisted breathing apparatus (1) according to claim 1, further comprises connections to the air/oxygen supplies already installed in medical units.

14. The assisted breathing apparatus (1) according to claim 1, wherein the humidification device (34) is a container that contains a portion of water with a temperature between 36 and 40° C., said temperature being achieved by thermal transfer from a set of resistive elements housed in a power supply (P) arranged on a side of the humidification device (34), or controlled by a resistive element.

15. The assisted breathing apparatus (1) according to claim 1, wherein the air/oxygen mixture outlet connection (40) internally includes an air quality sensor for monitoring CO₂ in the exhalation.

16. The assisted breathing apparatus (1) according to claim 1, wherein the microcontroller of the electronic board controls the opening and closing of the normal air solenoid valve (14) and the oxygen solenoid valve (23) through a dual-action relay, and also controls the CO₂ outlet solenoid valve (43) that manages the exhalation outlet, which is also controlled by said relay.

17. The assisted breathing apparatus (1) according to claim 1, wherein when the microcontroller determines, during the inspiration process, the closure of the CO₂ outlet solenoid valve (43), it opens the normal air solenoid valve (14) and the oxygen solenoid valve (23), allowing a pre-programmed amount of air and oxygen to pass to the patient.

18. The assisted breathing apparatus (1) according to claim 1, wherein during an exhalation process, the microcontroller closes the arrangement of the normal air solenoid valve (14) and the oxygen solenoid valve (23), and opens the CO₂ outlet solenoid valve (43).

19. The assisted breathing apparatus (1) according to claim 1, wherein further comprising a computer system configured to collect data and transmit it to a cloud environment and from there to a database, recombining and processing the data through algorithms and data science and artificial intelligence techniques.

20. The assisted breathing apparatus (1) according to claim 1, further comprising an audible and visual alarm system in case a confirmed parameter is out of range.

21. The assisted breathing apparatus (1) according to claim 1, wherein the normal air channel and the oxygen channel are supplied by air/oxygen sources already installed in medical units.

22. A respiratory assistance method characterized in that it comprises the steps of:
controlling the flow of regulated oxygen through an oxygen solenoid valve;
controlling the flow of regulated air through a normal air solenoid valve;
pre-establishing, through a plurality of graphical interfaces or user analog buttons, the values related to tidal volume, positive end-expiratory pressure, pressure trigger, inspired oxygen fraction, respiratory rate, and inspiration/expiration ratio according to the needs of the patient;
mixing the oxygen and air provided by the oxygen and air solenoid valves;
humidifying and increasing the temperature of the air/oxygen humidified mixture by device of a humidifier;
sensing the value related to the number of liters per minute (volume) of the humidified mixture through a flow sensor;
sensing the value related to the pressure of inspiration and expiration gases through the differential pressure sensor of the input/output;
conducting the humidified mixture at a pre-established pressure and volume to an air/oxygen outlet channel that conducts it toward a patient during the inspiration process;
monitoring the CO₂ value contained in the exhaled gases through an air quality sensor in the exhalation to establish;
automatically and in real time regulating, through a microcontroller, the oxygen pressure supplied to the patient, as well as the number of liters per minute (volume) of the humidified mixture;
wherein the step related to the regulation of oxygen and the number of liters per minute comprises the steps of:
controlling, by device of the air and oxygen solenoid valves, the flow and pressure of air and oxygen supplied to the patient in each breathing cycle from the pre-established values related to inspiration, plateau, and expiration, as well as the positive pressure at the end of expiration;
processing the sensed values related to the pressure of inspiration and expiration gases to determine in real time, based on said result, the actual pressure value of the humidified mixture supplied to the patient;
processing the sensed values related to the volume of the humidified mixture to determine in real time the actual volume of the humidified mixture supplied to the patient;
processing the sensed values related to the pressure of inspiration and expiration gases;
determining in real time, based on said result, the actual pressure value of the humidified mixture;
processing and determining the values related to the CO₂ content in the exhaled gases to define the opening and closing of a CO₂ outlet solenoid valve that allows the exit of the patient's exhaled gases;
determining in real time that the pre-established values correspond to the sensed values, which must be absolute and constant in order to maintain optimal respiratory conditions for the patient;
sending in real time at least one control signal to the On/Off solenoid valves of oxygen and/or air in order to compensate for at least one pressure difference between the programmed and the sensed value in each breathing cycle;
sending in real time at least one control signal to the pressure control solenoid valve to compensate for at least one difference between the programmed amount of liters of air per minute according to the patient's condition and the value sensed in each breathing cycle; and
sending in real time at least one control signal to the CO₂ outlet solenoid valve in case the sensed residual volume at the end of the expiration is lower than the pre-established value.

23. The respiratory assistance method according to claim 22, wherein when the patient reaches an inspiration point, the microprocessor closes the CO₂ outlet solenoid valve, opens the air solenoid valve and the oxygen solenoid valve, allowing a pre-programmed amount of air/oxygen to pass toward the patient, preventing pressure loss through the outlet, thereby ensuring that the air/oxygen mixture directed to the patient can only exit through the air/oxygen outlet toward the lungs.

24. The respiratory assistance method according to claim 22, wherein when the patient reaches an expiration point, the microprocessor closes the arrangement of the air solenoid valve and the oxygen solenoid valve and opens the CO₂ outlet solenoid valve, whereby the exhaled air exits through the CO₂ outlet and cannot be diverted or returned through the air/oxygen mixture conduction device.
